(19) United States
(12) Patent Application Publication
(10) Pub. No.: US 2025/0060356 A1
(43) Pub. Date: Feb. 20, 2025

(54) ARTIFICIAL BLOOD FOR REPRODUCING EXPECTORATED BLOOD SPATTER AND MANUFACTURING METHOD THEREOF

(71) Applicant: REPUBLIC OF KOREA (NATIONAL FORENSIC SERVICE DIRECTOR MINISTRY OF THE INTERIOR AND SAFETY), Wonju-si (KR)

(72) Inventors: Yong Jun KIM, Cheonan-si (KR); Young Il SEO, Wonju-si (KR); Jong Taek PARK, Wonju-si (KR); Kyu Young LIM, Wonju-si (KR)

(21) Appl. No.: 18/386,822

(22) Filed: Nov. 3, 2023

(30) Foreign Application Priority Data
Aug. 16, 2023 (KR) 10-2023-0107106

(51) Int. Cl. G01N 33/49

(52) U.S. Cl. CPC G01N 33/49

(57) ABSTRACT

Artificial blood for reproducing an expectorated blood spatter according to an embodiment includes maple syrup, water, and food coloring.

### CROSS-REFERENCE TO RELATED APPLICATIONS

The present application is based on and claims priority to Korean Patent Application No. 10-2023-0107106 filed on Aug. 16, 2023, in the Korean Intellectual Property Office, which is incorporated herein by reference in its entirety.

### BACKGROUND

### 1. Field

One or more embodiments relate to artificial blood for reproducing an expectorated blood spatter and a method of manufacturing the same.

### 2. Description of the Related Art

In criminal cases, bloodstain pattern analysis is important in estimating and reconstructing actions of a victim and a perpetrator at the time of the incident.

Forms of bloodstains found at crime scenes are very diverse, and these forms vary depending on the act causing bloodstain formation, the site of bleeding, and components of bloodstains. Bloodstains are classified and named based on the type of bloodstain.

When a bloodstain reproduction experiment is necessary during bloodstain pattern analysis, human blood, animal blood, or artificial blood is used. At this time, problems of user resistance and safety issues may arise. In particular, when using human blood and animal blood, there are many limitations in supplying the amount needed for experiments, as well as safety and ethical issues.

Artificial blood currently on the market for forensic science experiments has problems such as price burden, undisclosed product composition, and differences in physical characteristics from human blood.

Research on artificial blood is continuously being conducted to address the above problems. In particular, when reproducing an expectorated blood spatter, an experimenter needs to hold artificial blood in the mouth and then spit it out, so it is necessary to reduce resistance to artificial blood and ensure safety.

### SUMMARY

One or more embodiments include artificial blood capable of reducing user resistance when reproducing a bloodstain, such as an expectorated blood spatter, and that is not dangerous when ingested in a bloodstain reproduction experiment during bloodstain pattern analysis.

In addition, one or more embodiments include artificial blood that has physical properties and bloodstain shapes similar to those of human blood and existing artificial blood, allowing the trajectory of blood drops to be more accurately estimated when reconstructing a bloodstain shape.

According to one or more embodiments, artificial blood for reproducing an expectorated blood spatter may include maple syrup, water, and food coloring.

In an embodiment, a volume ratio of the maple syrup and the water is preferably 5:1.

In an embodiment, the food coloring may include red, yellow, and blue-violet tar colors.

In an embodiment, a mass ratio of the red, yellow and blue-violet tar colors is preferably 4:1:1.

According to one or more embodiments, a method of manufacturing artificial blood for reproducing an expectorated blood spatter may include making a maple syrup solution by mixing maple syrup and water, and mixing the maple syrup solution and food coloring.

In an embodiment, a volume ratio of the maple syrup and the water is preferably 5:1.

In an embodiment, the food coloring may include red, yellow, and blue-violet tar colors.

In an embodiment, a mass ratio of the red, yellow and blue-violet tar colors is preferably 4:1:1.

Additional aspects will be set forth in part in the description which follows and, in part, will be apparent from the description, or may be learned by practice of the embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other aspects will become apparent and more readily appreciated from the following description of the embodiments, taken in conjunction with the accompanying drawings in which:

FIG. 1 is a view of artificial blood for reproducing an expectorated blood spatter, according to an embodiment;

FIG. 2 is a flowchart illustrating a method of manufacturing artificial blood for reproducing an expectorated blood spatter, according to an embodiment;

FIG. 3 is a view of a falling bloodstain generator used in an experiment;

FIG. 4 is a view of photographs of the shape of a bloodstain generated by artificial blood for reproducing an expectorated blood spatter at each collision angle, according to an embodiment;

FIG. 5 is a data comparison graph for ratios of the short axis to the long axis of Example, human blood, and Comparative Examples 1 and 2;

FIG. 6 is a view illustrating a collision between a blood drop and a floor; and

FIG. 7 is a data comparison graph for estimated collision angle calculation results of Example, human blood, and Comparative Examples 1 and 2.

### DETAILED DESCRIPTION

It should be noted that in the following description, only parts necessary for understanding embodiments of the disclosure will be described, and descriptions of other parts will be omitted without departing from the scope of the disclosure.

The terms or words used in the present specification and claims described below should not be construed as being limited to ordinary or lexical meanings, and should be interpreted as meaning and concept corresponding to the technical idea of the disclosure based on the principle that the inventor may appropriately define the concept of a term in order to explain his or her invention in the best way. Therefore, because the configurations shown in the embodiments and drawings described in this specification are merely preferred embodiments of the disclosure and do not represent all of the technical idea of the disclosure, it should be understood that there may be various equivalents and modifications that can replace them at the time of this application.

Hereinafter, embodiments will be described in more detail with reference to the accompanying drawings.

In this specification, ‘expectorated blood spatter’ refers to a bloodstain spewing out from the victim's respiratory tract due to bleeding. To reproduce ‘expectorated blood spatter’, an experiment may be performed by holding artificial blood in the experimenter's mouth and then expelling it with force.

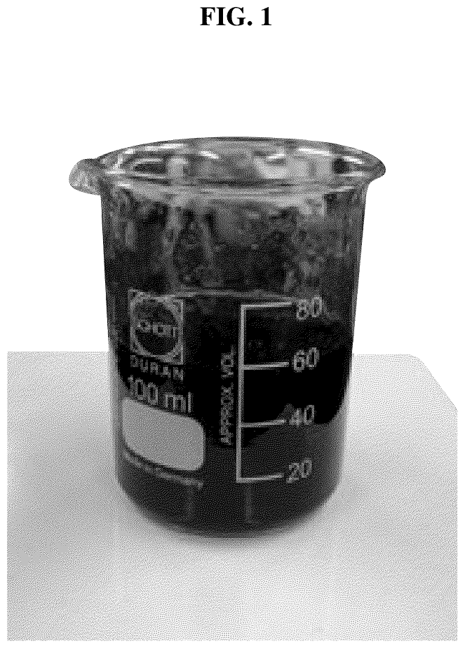

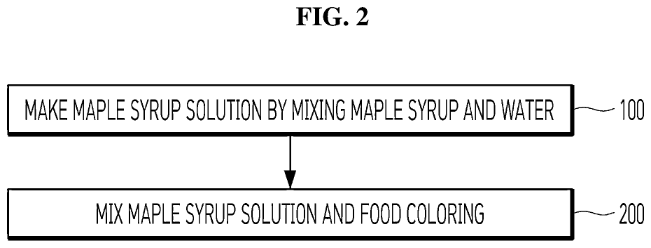

FIG. 1 is a view of artificial blood for reproducing an expectorated blood spatter, according to an embodiment. FIG. 2 is a flowchart illustrating a method of manufacturing artificial blood for reproducing an expectorated blood spatter, according to an embodiment.

Artificial blood for reproducing an expectorated blood spatter may include maple syrup, water, and food coloring.

In more detail, a method of manufacturing artificial blood for reproducing an expectorated blood spatter according to an embodiment is as follows.

Operation 100 is making a maple syrup solution by mixing maple syrup and water.

Maple syrup is a sweetener made from the sap of maple trees. Maple syrup contains water, sucrose, fructose and glucose, organic acids, amino acids, minerals, etc. Maple syrup is a viscous, edible, and food product that is easy to purchase. A volume ratio of the maple syrup and water is preferably 5:1.

Operation 200 is mixing the maple syrup solution and food coloring.

The food coloring may include tar colors. For example, the food coloring may include red, yellow, and blue-violet tar colors. A mass ratio of the red, yellow, and blue-violet tar colors is preferably 4:1:1.

By using food coloring like this, a color similar to human blood may be achieved. Edible tar coloring is used as a coloring agent, and although it is synthesized from benzene or naphthalene contained in coal tar, it has low toxicity and is water-soluble for safety reasons.

The artificial blood for reproducing an expectorated blood spatter manufactured above is stored in refrigeration and brought to a temperature similar to body temperature (approximately 37° C.) before use.

Hereinafter, the disclosure will be described in more detail by describing Example, Comparative Examples, and Experimental Examples. However, Example, Comparative Examples, and Experimental Examples are only examples of the disclosure, and the scope of the disclosure is not limited thereto.

### Example

A maple syrup solution is made by homogeneously mixing 50 ml of maple syrup and 10 ml of water (purified water). Food coloring is mixed homogeneously in the maple syrup solution at the ratio shown in Table 1.

TABLE 1

| Food | Tar | Tar | Tar |
|---|---|---|---|
| coloring | color | color | color |
| (color) | (red) | (yellow) | (blue-violet) |
| Ratio | 12 drops | 3 drops | 3 drops |

Here, 1 drop of tar color has a mass of approximately 1 g.

The artificial blood for reproducing an expectorated blood spatter of FIG. 1 is prepared using the above method.

### Comparative Example

Comparative Example 1 is artificial blood (NFBS) developed in advance by National Forensic Service, and Comparative Example 2 is SPATTER BLOOD (SB) from SIRCHIE, USA.

Example, human blood, and Comparative Examples 1 and 2 were tested by the method of Experimental Examples 1 and 2 below.

### Experimental Example 1: Viscosity Measurement

Viscosity is the resistance to the flow of a fluid. Viscosities of the prepared Example and Comparative Examples were measured using a viscometer.

Table 2 is a table showing viscosity measurement conditions according to an embodiment.

TABLE 2

| | Measurement | Measurement |
|---|---|---|
| Measurement | temperature | speed |
| element | (° C.) | (RPM) |
| condition | 36.5~37.0 | 30 |

Table 3 is a table showing viscosity measurement results of Example, human blood, and Comparative Example 1.

TABLE 3

| | Example | | |
|---|---|---|---|
| | (Artificial | | |
| | blood for | | |
| Viscosity | reproducing | Comparative | |
| (mPa · s, | expectorated | Example 1 | Human |
| cP) | blood spatter) | (NFBS) | blood |
| Result | 4.15~4.88 | 4.3 | 4.2 |

Referring to Table 3, viscosities of human blood and Comparative Example 1 (NFBS) are included within a range of measured viscosity of Example (artificial blood for reproducing an expectorated blood spatter).

### Experimental Example 2: Collision Angle Reproduction Experiment

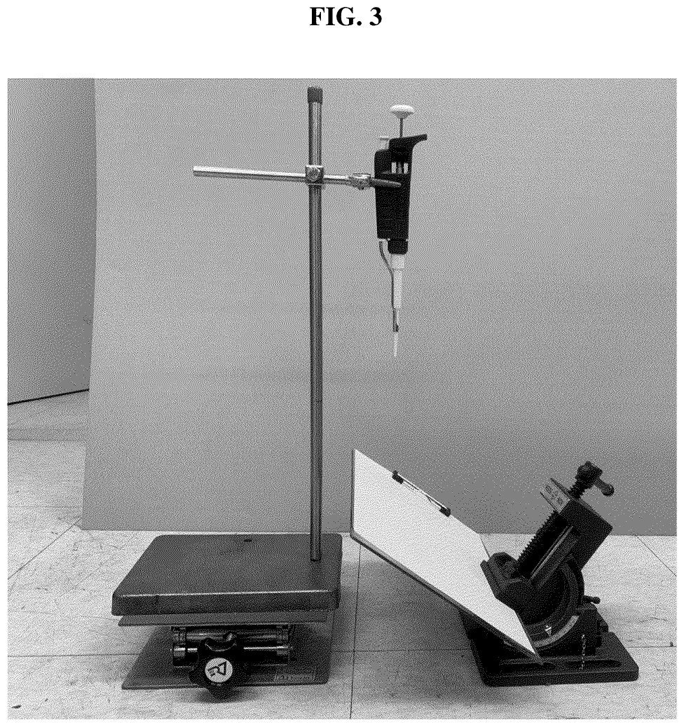

FIG. 3 is a view of a falling bloodstain generator used in an experiment. In this comparative experiment, each blood was tested under the same conditions of temperature 37° C., volume 20 μl, height 30 cm, and angles 20°, 30°, 40°, 50°, 60° and 70°, using the falling bloodstain generator of FIG. 3.

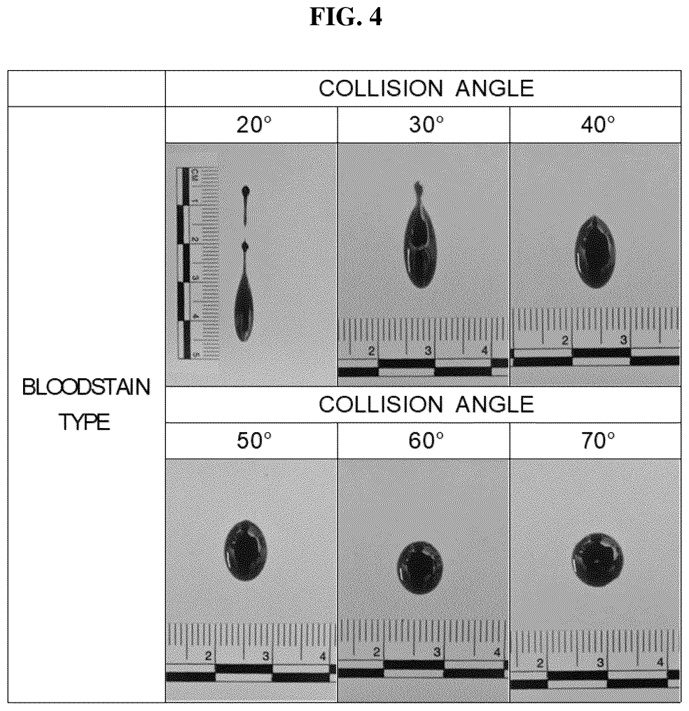

FIG. 4 is a view of photographs of the shape of a bloodstain generated by artificial blood for reproducing an expectorated blood spatter at each collision angle, according to an embodiment.

### Experimental Example 2-1: Measurement of a Ratio of the Short Axis to the Long Axis of a Bloodstain

Referring to FIG. 4, a ratio of the short axis W (Width) to the long axis L (Length) of a bloodstain generated by artificial blood for reproducing an expectorated blood spatter according to an embodiment is measured for each collision angle. For human blood and Comparative Examples 1 and 2, a ratio of the short axis to the long axis is measured from a bloodstain image.

Table 4 below shows data on ratios of the short axis to the long axis of Example, human blood, and Comparative Examples 1 and 2. Differences from values of Example are shown in each parenthesis of Table 4.

TABLE 4

| | Example | | | |
|---|---|---|---|---|
| | (Artificial | | | |
| | blood for | Com- | | Com- |
| | reproducing | parative | | parative |
| Collision | expectorated | Example 1 | Human | Example 2 |
| angle | blood spatter) | (NFBS) | blood | (SB) |
| 20° | 0.31 | 0.31(0.00) | 0.32(0.01) | 0.26(0.05) |
| 30° | 0.41 | 0.48(0.07) | 0.48(0.07) | 0.43(0.02) |
| 40° | 0.59 | 0.62(0.03) | 0.63(0.04) | 0.57(0.02) |
| 50° | 0.72 | 0.75(0.03) | 0.74(0.02) | 0.76(0.04) |
| 60° | 0.84 | 0.86(0.02) | 0.86(0.02) | 0.87(0.03) |
| 70° | 0.92 | 0.93(0.01) | 0.93(0.01) | 0.92(0.00) |

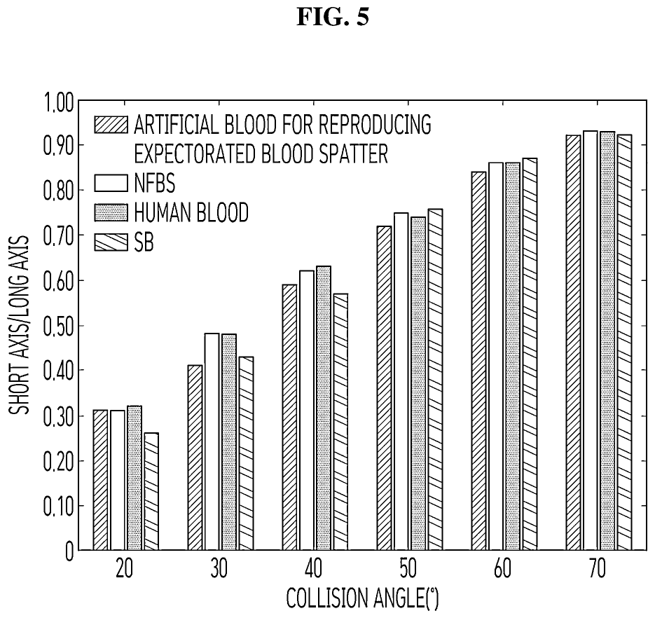

FIG. 5 is a data (Table 4) comparison graph for ratios of the short axis to the long axis of Example, human blood, and Comparative Examples 1 and 2.

Referring to values in each parenthesis in Table 4, a difference between the values of Example and values of Comparative Example 1 is 0.00 to 0.07, a difference between the values of the Example and values of human blood is 0.01 to 0.07, and a difference between the values of the Example and values of Comparative Example 2 is 0.00 to 0.05.

Accordingly, referring to Table 4 and FIG. 5, a ratio of the short axis to the long axis of artificial blood for reproducing an expectorated blood spatter according to Example is close to those of human blood and Comparative Examples 1 and 2.

### Experimental Example 2-2: Comparison of Calculation Results of an Estimated Collision Angle of a Bloodstain

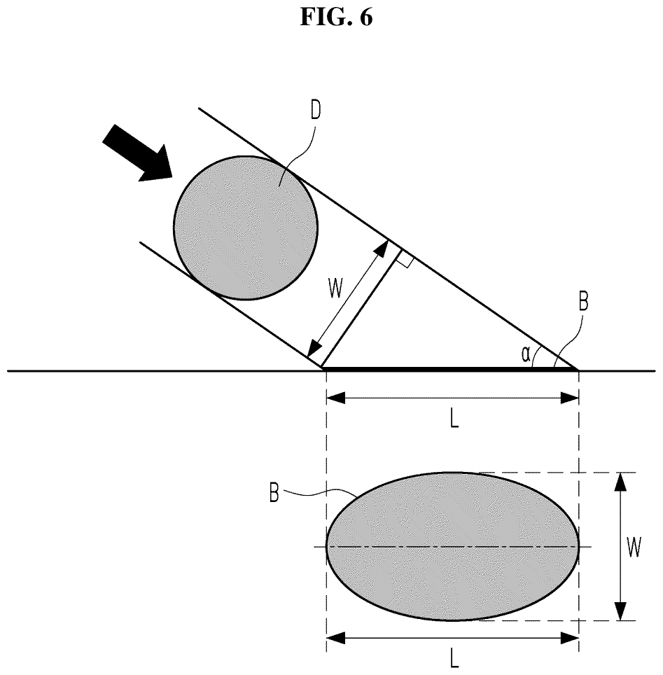

FIG. 6 is a view illustrating a collision between a blood drop and a floor. Referring to FIG. 6, a collision angle of a blood drop D may be expressed as Equation 1 below.

$$α = {sin}^{- 1} ( \frac{W}{L} ) \qquad [ Equation 1 ]$$

In Equation 1, a is a collision angle of the blood drop D, W is the short axis of a bloodstain B, and L is the long axis of the bloodstain B.

Referring to Equation 1 and FIG. 4, an estimated collision angle is calculated for a bloodstain at each collision angle generated by artificial blood for reproducing an expectorated blood spatter according to Example. For human blood and Comparative Examples 1 and 2, an estimated collision angle is also calculated from a bloodstain image. The estimated collision angle was calculated using HemoSpat software.

Table 5 below shows data on estimated collision angle calculation results for Example, human blood, and Comparative Examples 1 and 2. A difference from an actual collision angle (true value) is shown in each parenthesis in Table 5.

TABLE 5

| | Example | | | |
|---|---|---|---|---|
| | (Artificial | | | |
| | blood for | Com- | | Com- |
| Actual | reproducing | parative | | parative |
| collision | expectorated | Example 1 | Human | Example 2 |
| angle | blood spatter) | (NFBS) | blood | (SB) |
| 20° | 18.17°(1.83) | 18.00°(2.00) | 18.80°(1.20) | 15.10°(4.90) |
| 30° | 27.47°(2.53) | 28.00°(2.00) | 28.80°(1.20) | 25.80°(4.20) |
| 40° | 38.50°(1.50) | 39.00°(1.00) | 39.00°(1.00) | 34.50°(5.50) |
| 50° | 48.43°(1.57) | 48.90°(1.10) | 48.20°(1.80) | 49.30°(0.70) |
| 60° | 58.26°(1.74) | 58.80°(1.20) | 58.80°(1.20) | 60.60°(0.60) |
| 70° | 69.33°(0.67) | 68.10°(1.90) | 68.90°(1.10) | 69.40°(0.60) |

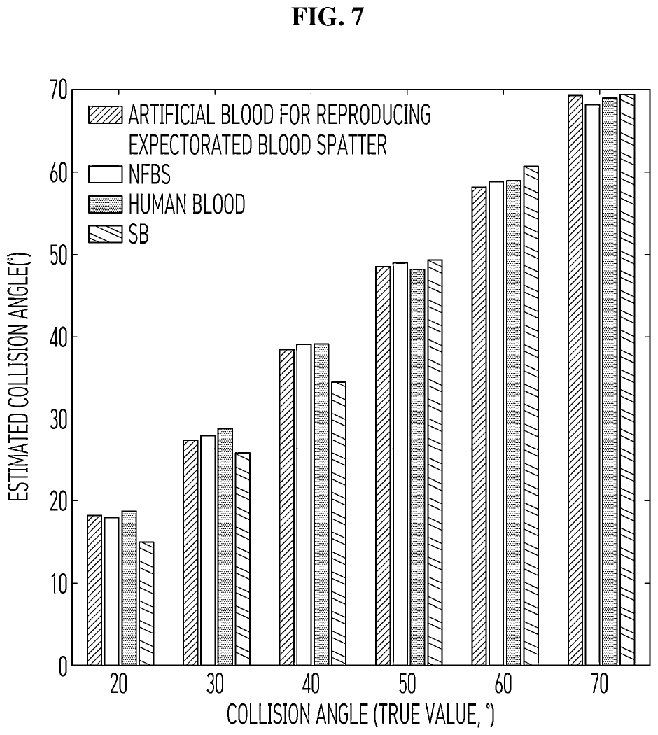

FIG. 7 is a data (Table 5) comparison graph for estimated collision angle calculation results of Example, human blood, and Comparative Examples 1 and 2.

Referring to values within each parenthesis in Table 5, a difference between the actual collision angles (true values) and values of Example is 0.67 to 2.53, a difference between the actual collision angles (true values) and values of Comparative Example 1 is 1.00 to 2.00, a difference between the actual collision angles (true values) and values of human blood is 1.00˜1.80, and a difference between the actual collision angles (true values) and values of Comparative Example 2 is 0.60 to 5.50.

Referring to Table 5 and FIG. 7, estimated collision angles for a bloodstain generated by artificial blood for reproducing an expectorated blood spatter according to Example have a smaller error range than that of the values of Comparative Example 2.

Accordingly, through the above Experimental Examples, it can be confirmed that artificial blood for reproducing an expectorated blood spatter has similar physical properties to those of human blood and existing artificial blood.

Because artificial blood for reproducing an expectorated blood spatter according to embodiments complements problems of human blood, animal blood, and existing artificial blood, it is safe for users to consume and has less resistance.

In addition, artificial blood for reproducing an expectorated blood spatter according to embodiments is implemented to have physical properties (viscosity, estimated collision angle, etc.) and a bloodstain shape similar to those of human blood and existing artificial blood, and thus, the trajectory of a blood drop may be estimated more accurately when reconstructing a bloodstain shape.

The artificial blood described above is not only applicable to reproducing an expectorated blood spatter, but can be applied to reproducing all types of bloodstains that a user experiments with by holding it in the mouth.

The embodiments disclosed in the present specification and drawings are merely presented as specific examples to aid understanding, and are not intended to limit the scope of the disclosure. It is obvious to one of ordinary skill in the art to which the disclosure pertains that other modifications based on the technical idea of the disclosure can be implemented in addition to the embodiments disclosed herein.

What is claimed is:

1. Artificial blood for reproducing an expectorated blood spatter comprising maple syrup, water, and food coloring.

2. The artificial blood for reproducing an expectorated blood spatter of claim 1, wherein a volume ratio of the maple syrup and the water is 5:1.

3. The artificial blood for reproducing an expectorated blood spatter of claim 1, wherein the food coloring comprises red, yellow, and blue-violet tar colors.

4. The artificial blood for reproducing an expectorated blood spatter of claim 3, wherein a mass ratio of the red, yellow and blue-violet tar colors is 4:1:1.

5. A method of manufacturing artificial blood for reproducing an expectorated blood spatter, the method comprising:
making a maple syrup solution by mixing maple syrup and water; and
mixing the maple syrup solution and food coloring.

6. The method of claim 5, wherein a volume ratio of the maple syrup and the water is 5:1.

7. The method of claim 5, wherein the food coloring comprises red, yellow, and blue-violet tar colors.

8. The method of claim 7, wherein a mass ratio of the red, yellow and blue-violet tar colors is 4:1:1.
